(19) United States
(12) Patent Application Publication
(10) Pub. No.: US 2026/0071194 A1
(43) Pub. Date: Mar. 12, 2026

(54) Method For Constructing Synthesis and Regeneration System Based on APS as Active Sulfonate Donor

(71) Applicant: Jiangnan University, Wuxi (CN)

(72) Inventors: Zhen KANG, Wuxi (CN); Jian CHEN, Wuxi (CN); Ruirui XU, Wuxi (CN); Guocheng DU, Wuxi (CN); Jianghua LI, Wuxi (CN); Yang WANG, Wuxi (CN)

(21) Appl. No.: 19/370,876

(22) Filed: Oct. 28, 2025

Related U.S. Application Data
(63) Continuation of application No. PCT/CN2024/088165, filed on Apr. 17, 2024.

(30) Foreign Application Priority Data
Apr. 28, 2023 (CN) 2023104797106
Apr. 28, 2023 (CN) 2023104864083

(51) Int. Cl. C12N 9/12; C12N 9/10; C12P 17/06; C12P 19/04; C12P 21/02

(52) U.S. Cl. CPC C12N 9/1229; C12N 9/1205; C12N 9/1241; C12N 9/13; C12P 17/06; C12P 19/04; C12P 21/02; C12Y 207/01025; C12Y 207/04001; C12Y 207/07004; C12Y 208/02005; C12Y 208/02008

(57) ABSTRACT

Disclosed is a method for constructing a synthesis and regeneration system based on APS as an active sulfonate donor, belonging to the technical field of biology. The present disclosure provides a new purpose of APS as an active sulfonate donor, and greatly improves the synthesis efficiency of the APS by screening different ATP sulfurylases and adding a pyrophosphatase into a reaction system to eliminate pyrophosphate as a byproduct. Further, the construction of a sulfonation modification system is realized by constructing an APS circulation regeneration system. Compared with a PAPS regeneration system, the APS circulation regeneration system has the advantages of short path and high efficiency, the sulfonation modification efficiency is significantly improved, and the synthesis cost is successfully reduced.

### REFERENCE TO SEQUENCE LISTING

The instant application contains a Sequence Listing in XML format as a file named “YGHY-2024-20-SEQ.xml”, created on October 27, 25, of 45,177 bytes in size, and which is hereby incorporated by reference in its entirety.

### TECHNICAL FIELD

The present disclosure relates to a method for constructing a synthesis and regeneration system based on APS as an active sulfonate donor, and belongs to the technical field of biology.

### BACKGROUND

Adenosine-5′-phosphosulfate (APS) is a precursor of 3′-phosphoadenosine-5′-phosphosulfate (PAPS). It is synthesized by catalyzing ATP and sulfate ions by an ATP sulfurylase (ATPS, EC 2.7.7.4), and pyrophosphate (PPi) as a byproduct is generated. It is generally considered that PAPS is the only universal active sulfonate donor. In our previous work, our research group speculated and verified that APS is also a universal active sulfonate donor. When being used as a novel active sulfonate donor, the APS has more advantages than PAPS. It has fewer reaction steps, and the byproduct AMP may be used for directly generating ATP through polyphosphate kinase, so that a regeneration path is shorter.

PAPS is in an active sulfonate form formed by activating sulfate ions by ATP through a two-step catalytic reaction via an ATP sulfurylase (ATPS, EC 2.7.7.4) and an APS kinase (APSK, EC 2.7.1.25), and is the only reported active sulfonate donor. Focusing on the major demands for people's life and health, various sulfotransferase libraries for polysaccharides (heparin, chondroitin sulfate), peptides (hirudin), flavonoids (naringenin), plant secondary metabolites (glucoside), hormones (estrogen), etc. have been mined and constructed; and in combination with PAPS catalysis circulation synthesis system modules, various cell-free sulfonation modification systems targeting target products have been constructed to achieve efficient biomanufacturing of various types of sulfonate compounds. However, due to the complex preparation process and high cost of PAPS, the synthesis progress of industrial sulfonic acid substances has been restricted, and it is unfavorable for large-scale production.

At present, for a PAPS regeneration cycle, sulfonate groups on PNPS are mainly transferred by a phenol sulfotransferase to 3′-adenosine phosphate-5′-phosphate (PAP) to synthesize PAPS. However, a byproduct PNP is a toxic substance, so it is unsuitable for food and medicine production. Therefore, it is of great significance to construct a green and non-toxic PAPS regeneration system to reduce the production cost, or construct a sulfonic acid modification system based on APS as a sulfonate donor to be applied to synthesis of various sulfo-group products such as polysaccharides (heparin, chondroitin sulfate), peptides (hirudin), flavonoids (naringenin), plant secondary metabolites (glucoside), and hormones (estrogen).

### SUMMARY

The present disclosure provides a method for regenerating adenosine-5′-phosphosulfate (APS). The APS is synthesized by using any one substance of AMP, ADP, or ATP as a substrate under the participation of a sulfotransferase, a polyphosphate kinase, and an ATP sulfurylase.

In an embodiment, the method uses the ATP and sulfate as substrates to synthesize the APS through catalysis.

In an embodiment, the amino acid sequence of the ATP sulfurylase is set forth in any one of SEQ ID NOs: 1-4.

In an embodiment, the amino acid sequence of the polyphosphate kinase is set forth in any one of SEQ ID NOs: 9-13.

In an embodiment, a pyrophosphatase also participates in the method, and the amino acid sequence of the pyrophosphatase is set forth in any one of SEQ ID NOs: 5-8.

In an embodiment, the method uses polyphosphate polyP_{n>2} and MgSO₄ as starting substrates.

In an embodiment, in a catalysis system for synthesizing APS through catalysis, a buffer solution is 10-50 mM Tris-HCl with a pH value of 6.0 to 8.0, added with 2 mM to 20 mM ATP, 1 mM to 5 mM magnesium sulfate, 10 mM to 200 mM sodium sulfate, and 0.5 mg/ml to 1.0 mg/mL ATP sulfurylase, sulfotransferase, and polyphosphate kinase.

In an embodiment, the sulfotransferase includes a chondroitin 4-O-sulfotransferase (the amino acid sequence is set forth in SEQ ID NO: 14), a heparin N-sulfotransferase (the amino acid sequence is set forth in SEQ ID NO: 15), a trehalose sulfotransferase (the amino acid sequence is set forth in SEQ ID NO: 13), or an estrogen sulfotransferase (the amino acid sequence is set forth in SEQ ID NO: 16).

In an embodiment, the sulfotransferase mutates based on a starting sequence. The mutation includes but is not limited to:
(1) mutating 143-position arginine into cysteine;
(2) mutating 152-position serine into proline; and
(3) mutating 154-position tryptophan into asparagine.

The present disclosure further provides an APS circulation regeneration system. In the APS circulation regeneration system, the APS may be catalyzed to synthesize AMP, the AMP may be catalyzed to synthesize ATP, and the ATP may be catalyzed to synthesize APS, so that a closed-loop cyclic catalytic reaction is formed. The catalytic reaction process is completed under the catalysis effect of an enzyme.

In an embodiment, the enzyme includes a sulfotransferase, a polyphosphate kinase, and an ATP sulfurylase.

In an embodiment, the enzyme is a sulfotransferase, a polyphosphate kinase, a pyrophosphatase, and an ATP sulfurylase.

In an embodiment, the sulfotransferase further has at least one of the following mutations:
(1) mutating 143-position arginine into cysteine;
(2) mutating 152-position serine into proline; and
(3) mutating 154-position tryptophan into asparagine.

In an embodiment, the amino acid sequence of the ATP sulfurylase is set forth in any one of SEQ ID NOs: 1-4; the amino acid sequence of the polyphosphate kinase is set forth in any one of SEQ ID NOs: 9-13; and the amino acid sequence of the pyrophosphatase is set forth in any one of SEQ ID NOs: 5-8.

In an embodiment, the APS circulation regeneration system uses cells as carriers.

In an embodiment, the cells include but are not limited to microbial cells capable of expressing at least one enzyme of the sulfotransferase, the polyphosphate kinase, and the ATP sulfurylase.

In an embodiment, the APS circulation regeneration system is a composition including the sulfotransferase, the polyphosphate kinase, and the ATP raising sulfurylase.

In an embodiment, the APS circulation regeneration system further includes polyphosphate polyP_{n>2} and sulfate, and n is an integer being greater than 2.

In an embodiment, the sulfate includes but is not limited to magnesium sulfate or sodium sulfate.

In an embodiment, the content of the sulfate is 2 mM to 200 mM.

The present disclosure further provides application of the APS circulation regeneration system to synthesis of a sulfonation product.

In an embodiment, the sulfonation product includes but is not limited to heparin, chondroitin sulfate, hirudin, or flavone.

In an embodiment, the catalytic reaction temperature is 20° C. to 40° C., and the catalysis time is 24 h to 72 h.

The present disclosure provides application of adenosine-5′-phosphosulfate (APS) as a novel general active sulfonate donor.

In an embodiment, the application refers to application of a conversion reaction from the APS to the AMP to a catalytic production process of a sulfonate compound.

The present disclosure further provides application of the method to increase of regeneration circulation times of adenosine-5′-phosphosulfate or synthesis of a sulfonate product.

The present disclosure further provides a PAPS regeneration circulation system, including a polyphosphate kinase, a PAP dephosphorylation enzyme, and a PAPS synthesis bifunctional enzyme, being applicable to PAPS synthesis through catalysis by one or more of the above enzymes from any one substance of AMP, ADP, ATP, APS, or PAP, and participating in catalyzing module I, module II, and module III forming a closed-loop cycle. module I converts the AMP into the ATP, module II converts the ATP into the PAPS, and module III converts the PAPS into the AMP.

In an embodiment, the enzyme includes a polyphosphate kinase, a PAP dephosphorylation enzyme, a sulfotransferase, and a PAPS synthesis bifunctional enzyme.

In an embodiment, module I catalyzes the AMP through a polyphosphatase to produce the ATP.

In an embodiment, the polyphosphate kinase has ADP and AMP phosphorylation bifunctional activity.

In an embodiment, the polyphosphate kinase has the amino acid sequence set forth in any one of SEQ ID NOs: 8-12.

In an embodiment, module II catalyzes the ATP through a PAPS synthesis bifunctional enzyme to synthesize the PAPS.

In an embodiment, the PAPS synthesis bifunctional enzyme may be replaced with a combination of an APS kinase and an ATP sulfurylase from different sources.

In an embodiment, module Ill catalyzes the PAPS through a PAP dephosphorylation enzyme and a sulfotransferase to produce the AMP.

In an embodiment, the PAP dephosphorylation enzyme has the amino acid sequence set forth in any one of SEQ ID NOs: 22-25.

In an embodiment, the PAPS regeneration circulation system further includes sulfotransferase, polyphosphate polyP_{n>2}, and sulfate.

In an embodiment, the sulfotransferase includes but is not limited to a chondroitin 4-O-sulfotransferase C4ST, a heparin N-sulfotransferase, a trehalose sulfotransferase Stf0, or an estrogen sulfotransferase EST.

In an embodiment, the sulfate includes but is not limited to magnesium sulfate or sodium sulfate.

In an embodiment, the content of the sulfate is 2 mM to 200 mM.

In an embodiment, in a catalysis system for synthesizing 3′-phosphoadenosine-5′-phosphosulfate through catalysis, a buffer solution is 10-50 mM Tris-HCl with a pH value of 6.0 to 8.0, added with 2 mM to 20 mM ATP, 1 mM to 5 mM magnesium sulfate, and 0.5 mg/ml to 1.0 mg/ml artificial PAPS synthesis bifunctional enzyme.

In an embodiment, the PAPS regeneration circulation system is a composition of the polyphosphate kinase, the PAP dephosphorylation enzyme, and the PAPS synthesis bifunctional enzyme.

In an embodiment, the PAPS regeneration circulation system uses cells as carriers.

In an embodiment, the cells include but are not limited to microbial cells capable of expressing at least one enzyme of the polyphosphate kinase, the PAP dephosphorylation enzyme, and the PAPS synthesis bifunctional enzyme.

In an embodiment, the PAPS regeneration circulation system is applied in a form of a complex enzyme preparation.

In an embodiment, the content of the polyphosphate kinase, the PAP dephosphorylation enzyme, and the PAPS synthesis bifunctional enzyme in the complex enzyme preparation is greater than or equal to 0.1 g/L.

The present disclosure further provides application of the PAPS regeneration circulation system to production of a sulfonation product.

In an embodiment, the application includes but is not limited to synthesis of heparin, chondroitin sulfate, hirudin, or flavone.

In an embodiment, the catalytic reaction temperature is 20° C. to 40° C., and the catalysis time is 24 h to 72 h.

The present disclosure further provides a method for regenerating 3′-phosphoadenosine-5′-phosphosulfate, or application of the method to increase of regeneration circulation times of the 3′-phosphoadenosine-5′-phosphosulfate or synthesis of a sulfonate product.

The present disclosure provides a method for synthesizing PAPS, including: synthesizing 3′-phosphoadenosine-5′-phosphosulfate through catalysis by using a PAPS synthesis bifunctional enzyme and by using ATP and sulfate as substrates.

### Beneficial Effects

1. By verifying the APS as a novel active sulfonate donor, the present disclosure realizes shorter-step sulfonate donor synthesis and shorter-step regeneration, greatly reduces the cost of a sulfonation modification reaction, and successfully completes the sulfonation modification synthesis on heparin, chondroitin sulfate, trehalose sulfonate, etc.
2. By adding a hydrolase of pyrophosphate as a byproduct into an APS synthesis system, the present disclosure greatly improves the synthesis efficiency of the APS.
3. By modifying a 3′PB motif (3′-phosphate binding site) in sulfotransferase, the present disclosure improves the affinity of the sulfotransferase to the APS, improves the utilization of the APS, and further improves the sulfonation modification efficiency of the APS regeneration circulation.
4. By designing the PAPS regeneration path, the present disclosure simplifies the catalysis steps, realizes the construction of a PAPS regeneration system based on cheap substrates of polyphosphate and MgSO₄, improves the sulfonation modification efficiency, is successfully applied to sulfonation modification synthesis of heparin, chondroitin sulfate, trehalose sulfonate, etc., and greatly reduces the PAPS synthesis application cost. The cost is reduced by half after each cycle.
5. By designing module I of the PAPS regeneration path, the present disclosure realizes one-step synthesis of ATP from AMP, simplifies the catalysis steps, improves the synthesis efficiency, and is hopeful to be used in various ATP regeneration systems.
6. By designing module III of the PAPS regeneration path, and mining PAP dephosphorylation enzymes from different sources, the present disclosure relieves the feedback inhibition of the byproduct PAP on the sulfotransferase, improves the enzyme activity of the sulfotransferase, and improves the sulfonation modification efficiency.
7. By applying the PAPS regeneration system built by combining all the modules to application of chondroitin sulfate A and heparin, the present disclosure realizes the determination of cycle times exceeding 4 times, and greatly reduces the synthesis cost.

### BRIEF DESCRIPTION OF FIGURES

FIG. 1 shows a schematic diagram and module division of a PAPS regeneration system.

FIG. 2 shows specific enzyme activities of polyphosphate kinases in module I.

FIG. 3 shows application of a polyphosphate kinase to the PAPS synthesis system.

FIG. 4A-4B shows PAP dephosphorylation enzymes from different sources, and application to chondroitin sulfate A sulfonation modification.

FIG. 5 shows enzyme activity determination of sulfotransferases: a trehalose sulfotransferase and an estrogen sulfotransferase.

FIG. 6 shows application of the PAPS regeneration system to modification of chondroitin sulfate synthesis.

FIG. 7 shows application of the PAPS regeneration system to modification of heparin synthesis.

FIG. 8 shows application of the PAPS regeneration system to modification of trehalose-2-sulfonate and estrogen sulfonate synthesis.

FIG. 9 shows structure differences between APS and PAPS.

FIG. 10 shows modification and synthesis of a sulfonation product based on APS as a sulfonate donor.

FIG. 11 shows specific enzyme activities of ATP sulfurylases from different sources.

FIG. 12 shows specific enzyme activities of pyrophosphatases from different sources.

FIG. 13 shows a schematic diagram of an APS circulation regeneration system.

FIG. 14 shows specific activities of polyphosphate kinases from different sources.

FIG. 15 shows circulation times of the APS circulation regeneration system applied to sulfonate product modification.

FIG. 16 shows 3′PB spatial locations of different sulfotransferases.

FIG. 17 shows relative enzyme activities of a 3′PB-position mutant of a trehalose sulfotransferase.

### DETAILED DESCRIPTION

1. E. BL21 (DE3) was a commercial strain, and pET28a (+) was a commercial plasmid.
2. Plasmid construction reagents were purchased from Sangon Biotech (Shanghai) Co., Ltd., and the sequencing verification was completed in Sangon Biotech (Shanghai) Co., Ltd.
3. Various analytical reagents were purchased from China National Pharmaceutical Group Co., Ltd. (Sinopharm), and APS (analytical grade) was purchased from Sigma.
4. Culture medium:
LB Culture: 10 g/L NaCl, 10 g/L tryptone, and 5 g/L yeast powder.
TB Culture: 2.31 g/L KH₂PO₄, 12.54 g/L K₂HPO₄, 12 g/L tryptone, 24 g/L yeast powder, and 4 mL/L of glycerol.
Protein expression: Recombinant plasmids containing encoding target protein genes were transfected into E. coli BL21 (DE3). After verification, streak culture was performed on a plate containing a corresponding antibiotic. A single colony was picked to be inoculated into an LB seed culture. After culturing to acquire a seed solution, the seed solution was transferred and inoculated into a 50 mL TB fermentation medium according to a volume fraction of 1 mL/50 mL. Culture was continuously performed for 1 h to 2 h, then, 0.5 mM IPTG was added, and induction culture was performed at 30° C. for 10 h. After the completion, cells were collected, and were used for purification and analysis after ultrasonic disruption.
Protein purification: After ultrasonic disruption, the collected cells were subjected to high-speed centrifugation to remove cell debris. The supernatant was filtered by a 0.22 μm water-compatible filter membrane, and target protein was purified by using Ni-NTA affinity chromatography. After the column was equilibrated with a solution A, a crude enzyme was loaded. Then, a chromatographic column was equilibrated with the solution A. Next, the chromatographic column was eluted with a solution B of different concentrations, and elution fractions were collected. Purified components were verified by SDS-PAGE. The purest components were desalinized through a PD-10 desalinizing column. During desalinization, a low-salt buffer solution (10 mM Tris-HCl, 0.1 M NaCl; pH 6.0) was used, and purified desalinized protein was obtained through collection. Solution A: 20 mM Tris-HCl buffer with a pH value of 7.5, and 500 mM NaCl. Solution B: 20 mM Tris-HCl buffer with a pH value of 7.5, 500 mM NaCl, and 500 mM imidazole.
5. Detection method:
AMP, ADP, ATP, and APS detection methods: The followings were adopted: an Agilent 1600 HPLC system, a Polyamine II column (4.6×250 mm, 12 nm), a mobile phase: 50 mM KH₂PO₄ and 0.1% triethylamine solution, a flow velocity: 0.6 mL·min⁻¹, a sample size: 5 μL, detection time: 35 min, and a detector: UV 254 nm.
Enzyme activity of sulfotransferase: An enzyme amount of the corresponding sulfotransferase required for transferring 1 mM sulfonate groups onto a target product per hour under the condition of 35° C.
Circulation times: The circulation times mentioned in the specific embodiment referred to the utilization times of the added substrate in the whole circulation system, for example, the utilization times of the substrate APS in the whole APS circulation system, with a calculation method of dividing an amount of products in the circulation system by an amount of products in catalysis initially added into the APS; and the utilization times of the substrate ATP in the whole PAPS circulation system.
Definition of enzyme activity of ATP sulfurylase: An enzyme amount required for degradation of 1 μM APS per hour under the condition of 35° C.
Definition of enzyme activity of PAP dephosphorylation enzyme: An enzyme amount required for degradation of 1 μM PAP per hour under the condition of 35° C.
Definition of enzyme activity of ADP phosphorylase: An enzyme amount required for generation of 1 μM PAP per minute by using the ADP as a substrate under the condition of 35° C.
Definition of enzyme activity of AMP phosphatase: An enzyme amount required for generation of 1 μM ADP per minute by using the AMP as a substrate under the condition of 35° C.

### Example 1: Screening of Polyphosphate Kinase (Module I)

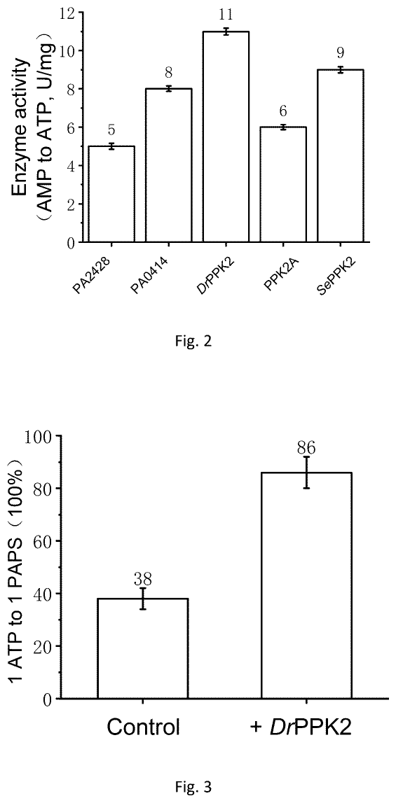

Enzymes capable of fast converting AMP into ATP were screened: Enzymes (set forth in SEQ ID NO: 8 and SEQ ID NO: 9) from a source of Pseudomonas aeruginosa, an enzyme (Genbank ID: WP_011013972.1) from a source of Corynebacterium glutamicum, an enzyme (SePPK2, CDD: 274736) from a source of Staphylococcus epidermidis, and an enzyme (DrPPK2, CDD: 274737) from a source of Deinococcus radiodurans were respectively selected, and the amino acid sequences were set forth in SEQ ID NOs: 8-12. Codon optimization was performed according to an E. coli codon preference law to respectively obtain gene sequences set forth in SEQ ID NOs: 26-28, 17-18. The optimized nucleotide sequences were respectively ligated into the plasmid pET28a (+) between Nde I and Hind III restriction sites to respectively obtain recombinant plasmids. The recombinant plasmids were expressed in E. coli BL21 (DE3), and were cultured in a TB Culture at 30° C. till OD=1.0, and induced fermentation was performed by using 0.5 mM IPTG under the condition of 30° C. Intracellular enzymes were collected, and the specific enzyme activity was measured after purification. The result is shown in FIG. 2. The above polyphosphate kinase has the activities of the AMP phosphorylase and the ADP phosphorylase at the same time, can be used for one-step direct synthesis of ATP from AMP by using polyP_{n>2} as a phosphate donor, and can also be applicable to PAPS synthesis and other reactions requiring ATP synthesis.

### Example 2: Application of Polyphosphate Kinase to Production of PAPS

The polyphosphate kinase screened in Example 1 was used for regeneration of byproducts in a PAPS synthesis process.

In a PAPS synthesis system, the followings were included: 50 mM Tris-HCl with a pH value of 7.5, 5 g/L ATP, 20 mM MgSO₄, 0.5 g/L PAPS synthesis bifunctional enzyme, and 0.5 g/L polyphosphate kinase zymoprotein DrPPK2 prepared in Example 1. The PAPS synthesis bifunctional enzyme might be replaced with a combination of an APS kinase and an ATP sulfurylase from different sources.

By taking a reaction system in the absence of polyphosphate kinase as a control, the result is shown in FIG. 3. In the reaction system of this example, the conversion rate of the ATP to the PAPS is improved from 36% to 86%. It proves that the recovery on byproducts in PAPS synthesis is realized by using DrPPK2.

Optionally, the DrPPK2 zymoprotein in the PAPS synthesis system of this example might further be replaced with recombinant microbial cells having the same zymoprotein expression amount and expressing the same enzyme, and the similar effect might be acquired.

### Example 3: Screening of PAP Dephosphorylation Enzyme (Module III)

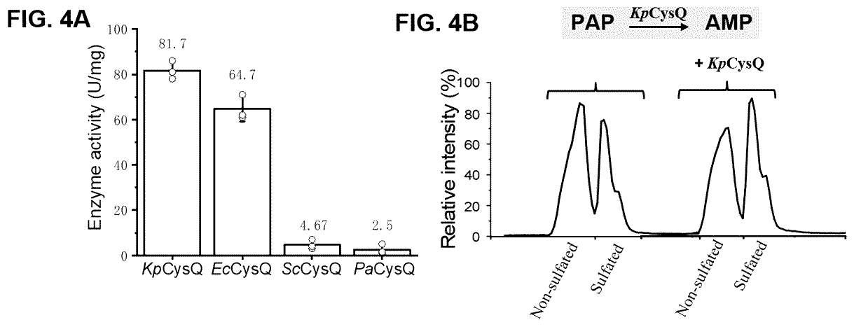

After the active group transfer from the PAPS, PAP as a byproduct might be generated, the PAP might continuously occupy a substrate binding pocket of the sulfotransferase, and might thus influence the activity of the sulfotransferase. By screening the PAP dephosphorylation enzyme, the byproduct PAP was degraded, and the activity of sulfatase and the utilization of PAPS were improved. The PAP dephosphorylation enzymes from different sources, including but not limited to E. coli (EcCysQ, amino acid sequence set forth in SEQ ID NO: 22, and gene sequence as shown by Gene ID: 948728), P. aeruginosa (PaCysQ, amino acid sequence set forth in SEQ ID NO: 23), Pichia pastoris (KpCysQ, amino acid sequence set forth in SEQ ID NO: 24), and Saccharomyces cerevisiae (ScCysQ, amino acid sequence set forth in SEQ ID NO: 25), were compared and screened. Gene segments (set forth in SEQ ID NOs: 29-32) encoding the above PAP dephosphorylation enzyme were respectively synthesized, and were ligated into plasmids pET28a (+) between Nde I and Hind III restriction sites to obtain recombinant plasmids. The recombinant plasmids were expressed in E. coli BL21 (DE3), and were cultured in a TB Culture at 30° C. till OD=1.0, and induced fermentation was performed by using IPTG with a final concentration of 0.5 mM under the condition of 30° C. Intracellular enzymes were collected, purification was performed, and the specific enzyme activity was measured (FIG. 4A). The result shows that the EcCysQ from the source of E. coli and the KpCysQ from the source of P. pastoris have the activity of the PAP dephosphorylation enzyme, and the values are respectively 64.7 U/mg and 81.7 U/mg.

The PAP dephosphorylation enzyme was added into a catalytic reaction system (50 mM Tris-HCl, pH=7.5) containing 2.0 g/L chondroitin sulfate A to react for 12 h under the condition of 30° C. An amount of a corresponding sulfonation product was detected. The result shows that the activity of the sulfotransferase is successfully improved, and the peak area increase of the sulfonation product is obviously seen from FIG. 4B.

### Example 4: Activity Expression of Sulfotransferases: Trehalose Sulfotransferase and Estrogen Sulfotransferase

The chondroitin 4-O-sulfotransferase (C4ST) was prepared according to a method in CN106244566A, and the heparin N-sulfotransferase (NST) was prepared according to a method in CN107384990A.

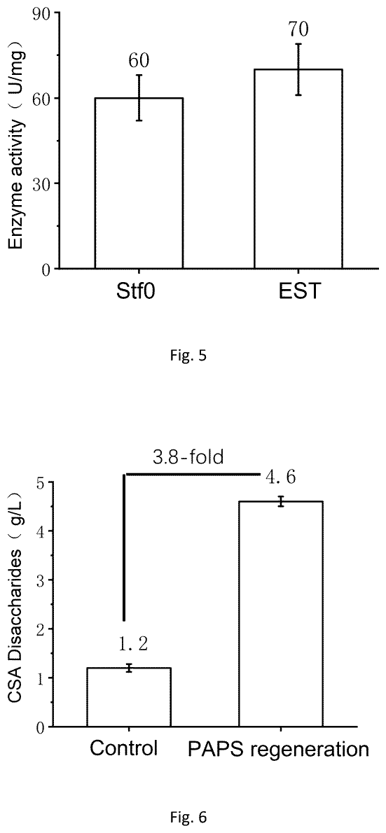

Through the screening of trehalose sulfotransferase (Gene ID: 66738808, Stf0, amino acid sequence set forth in SEQ ID NO: 13) and estrogen sulfotransferase (Gene ID: 360268, EST, amino acid sequence set forth in SEQ ID NO: 16), and through E. coli codon optimization, gene segments set forth in SEQ ID NO: 19 and SEQ ID NO: 21 were respectively obtained. The optimized gene segments were ligated into the plasmid pET28a (+) between Nde I and Hind III restriction sites, and were transferred into E. coli BL21 (DE3). After streak on a plate, a single colony was picked to culture a seed solution. The seed solution was transferred and inoculated into a shake flask at a concentration of 2%. Culture was continuously performed for 2 h at 30° C. 0.5 mM IPTG was added, and induction was continuously performed at 30° C. for 15 h to obtain a crude enzyme solution of sulfotransferase. The zymoprotein was purified, and activity analysis was performed. As shown in FIG. 5, the enzyme activity of the trehalose sulfotransferase Stf0 is 60 U/mg, and the enzyme activity of the estrogen sulfotransferase EST is 70 U/mg.

### Example 5: Design and Module Division of PAPS Regeneration Circulation System

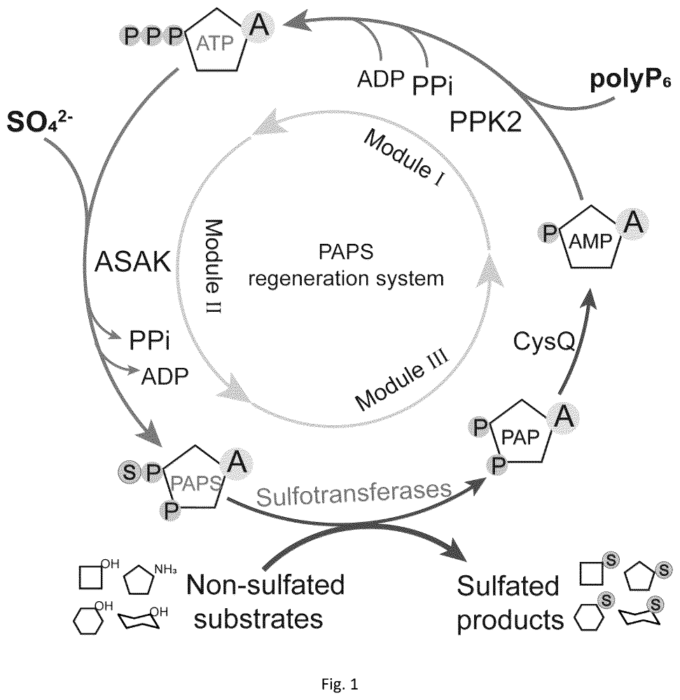

Based on the PAPS synthesis bifunctional enzyme ASAK disclosed in CN113046402A, and a method for one-step PAPS synthesis from ATP, in combination with reaction free energy calculation, chemometry analysis and byproduct diversion strategies, a PAPS regeneration system (FIG. 1) was designed. Through screening, expression and verification of a PAP esterase, an AMP phosphorylase, an ADP phosphorylase, the PAPS regeneration system was successfully constructed. Through the construction sequence, the PAPS regeneration system was organically divided into three modules to be respectively verified and spliced.

(1) Construction of module I: According to the method in Example 1, the polyphosphate kinase successfully converting AMP into ATP was successfully screened. A catalysis system using the AMP as the substrate and added with polyphosphate and polyphosphate kinase was constructed. Components of the catalysis system might include: 50 mM Tris-HCl with a pH value of 7.5, 10 mM AMP, 20 mM polyP₆, and 0.2-1.0 g/L SePPK2. The catalysis system might realize the conversion from AMP to ATP.

(2) Construction of module II: A catalysis system using the ATP as the substrate and added with sulfate and a PAPS synthesis bifunctional enzyme was constructed. Components of the catalysis system might include: 50 mM Tris-HCl with a pH value of 7.5, 10 mM ATP, 20 mM MgSO₄, and 0.5 g/L PAPS synthesis bifunctional enzyme ASAK.

The PAPS synthesis bifunctional enzyme might be an enzyme disclosed in CN113046402A, or might be a combination of an ATP sulfurylase and an APS kinase, including but not limited to an ATP sulfurylase from a source of S. cerevisiae and an APS kinase from a source of E. coli.

(3) Construction of module III: A PAP dephosphorylation enzyme with the activity was successfully screened, and was used for byproduct PAP elimination and AMP regeneration. A catalysis system using the PAPS as the substrate and added with the PAP dephosphorylation enzyme and a sulfotransferase was constructed. Components of the catalysis system might include: 50 mM Tris-HCl with a pH value of 7.5, 10 mM ATP, 20 mM MgSO₄, and 0.5 g/L PAP dephosphorylation enzyme cysQ.

The PAP dephosphorylation enzyme might be EcCysQ with the amino acid sequence set forth in SEQ ID NO: 22 or KpCysQ with the amino acid sequence set forth in SEQ ID NO: 24. The sulfotransferase might be a chondroitin 4-O-sulfotransferase C4ST disclosed by CN106244566A, a heparin N-sulfotransferase disclosed by CN107384990A, a trehalose sulfotransferase Stf0 with the amino acid sequence set forth in SEQ ID NO: 13, or an estrogen sulfotransferase EST with the amino acid sequence set forth in SEQ ID NO: 16.

A working principle of the PAPS regeneration system was as follows: As shown in FIG. 1, the PAPS regeneration system constructed in this example might be used for generating a corresponding product through an enzymic catalytic reaction from any one substance of AMP, ADP, ATP, APS, or PAP. Through the low-cost supply of polyphosphate and sulfonate groups, the reaction system might realize the energy circulation regeneration. The sulfonate groups might be provided through a sulfonation modification reaction, that was, through active sulfonate donor transfer without the consumption on frameworks (AMP structure portions), the framework circulation was realized, sulfonate ions were activated in the circulation process, and the supply of the active sulfonate groups was realized, so that the supply of the active sulfonate groups might be realized only through the external supply of sulfate ions and polyphosphate.

### Example 6: Application of PAPS Regeneration System to Production of Chondroitin Sulfate

A system (0.5 g/L PPK2, 0.5 g/L ASAK, 0.5 g/L CysQ, 20 mM polyP₆, 10 mM ATP, and 50 mM MgSO₄) of the PAPS regeneration circulation system was added into the reaction system. 0.5 g/L chondroitin 4-O-sulfotransferase C4ST (prepared according to a method in CN106244566A) and 5 g/L chondroitin substrate metered according to a final concentration were added. Catalysis was performed at 35° C. for 6 h. The generation of a sulfonation product was detected by a liquid chromatography-mass spectrometry technology.

A control group was set. The control group adopted an existing conventional catalysis production manner. A catalysis system included 0.5 g/L ASAK, 10 mM ATP, and 50 mM MgSO₄. 0.5 g/L chondroitin 4-O-sulfotransferase C4ST (prepared according to a method in CN106244566A) and 5 g/L chondroitin substrate were added, and catalysis was performed at 35° for 6 h.

Through analysis on the substrate, the yield of the chondroitin sulfate A produced by using the PAPS circulation regeneration system is 4.6 g/L, but the yield in the control group is only 1.2 g/L, and the yield of the product was increased to 3.8 times. That is, the circulation times of the PAPS regeneration system is 3.8 (as shown in FIG. 6).

### Example 7: Application of PAPS Regeneration Circulation System to Production of Heparin

A system (0.5 g/L PPK2, 0.5 g/L ASAK, 0.5 g/L CysQ, 20 mM polyP₆, 10 mM ATP, and 50 mM MgSO₄) of the PAPS regeneration circulation system was added into the reaction system. 0.5 g/L heparin N-sulfotransferase NST (prepared according to a method in CN107384990A) and 5 g/L deacetylated heparin substrate were added. Catalysis was performed at 35° C. for 6 h, and the generation of a sulfonation product was detected by a liquid chromatography-mass spectrometry technology.

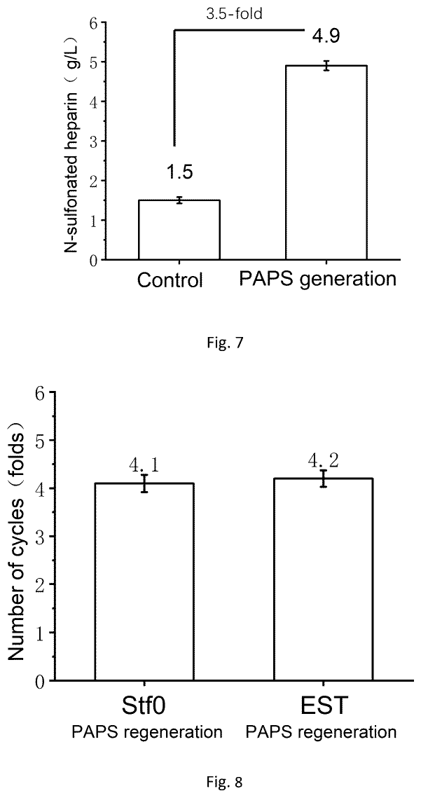

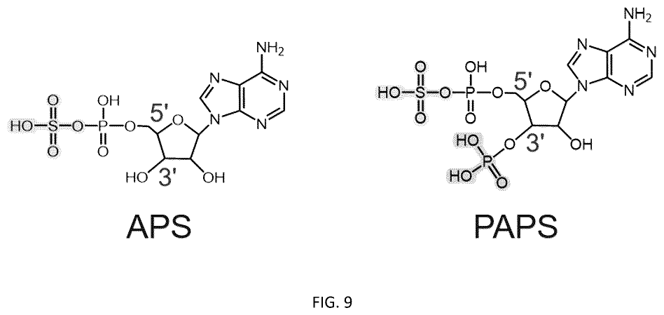

A control group was set. The control group adopted an existing conventional catalysis production manner. 0.5 g/L heparin N-sulfotransferase NST (prepared according to a method in CN107384990A) and 5 g/L deacetylated heparin substrate were added. Catalysis was performed at 35° C. for 6 h. Through analysis on the substrate, the yield of N-sulfated heparin modified through reaction by using the PAPS circulation regeneration system is 4.9 g/L, but the yield in the control group is only 1.4 g/L, and the yield of the product is increased to 3.5 times. That is, the circulation times of the PAPS regeneration system are 3.5 (as shown in FIG. 7).

### Example 8: Application of PAPS Regeneration System to Production of Trehalose-2-Sulfonate and Estrogen Sulfonate

This example differed from the method of Example 7 in that: the sulfotransferase was respectively replaced with a trehalose sulfotransferase and an estrogen sulfotransferase, and the substrate was respectively replaced with trehalose and estrogen. Catalysis was performed at 35° C. for 6 h, The generation of a sulfonation product was detected by a liquid chromatography-mass spectrometry technology.

The results are shown in FIG. 8, and the circulation times of the corresponding PAPS regeneration system are of 4.1 and 4.2.

### Example 9: Preparation of PAPS Regeneration System in Enzyme Preparation Form

A PAPS catalysis system was prepared from 50 mM Tris-HCl, 0.5 g/L PPK2, 0.5 g/L ASAK, 0.5 g/L CysQ, 20 mM polyP₆, 10 mM ATP, and 50 mM MgSO₄. Firstly, PPK2, ASAK, and CysQ were expressed in E. coli. Affinity chromatography purification was performed to obtain a pure enzyme. After quantitation, the pure enzyme was put into 50 mM Tris-HCl to obtain a mixed enzyme preparation. A substrate (polyP₆, ATP, and MgSO₄) and a corresponding sulfotransferase might be added in use.

### Example 10: Preparation of PAPS Regeneration System of Immobilized Enzyme

Self-aggregating assembly proteins cipA were added to the N-terminal of PPK2, ASAK, and CysQ. Automatic sedimentation occurred during expression to form active inclusion bodies. Proteins might be obtained through centrifugation. The three kinds of proteins were combined to be added into 50 mM Tris-HCl to obtain a mixed enzyme preparation. A substrate (polyP₆, ATP, and MgSO₄) and a corresponding sulfotransferase might be added in use. After the catalysis was completed, centrifugation was performed again, and enzyme components were collected for next catalysis.

### Example 11: Application of APS as Active Sulfonate Donor

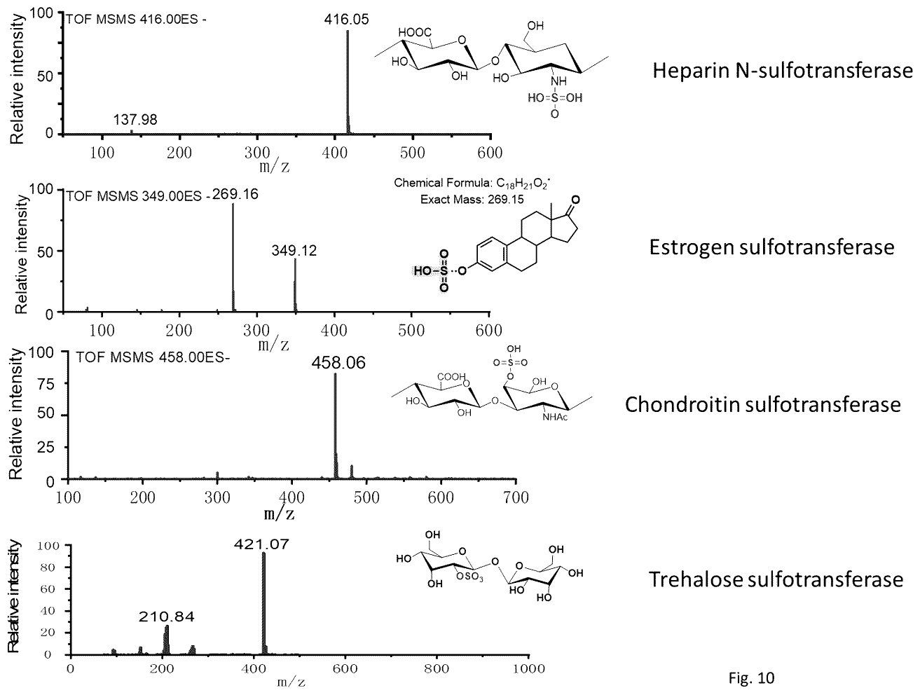

APS was a PAPS synthetic precursor, and they only had a difference of a phosphate group in a 3′ position. In order to verify that the APS might be used as a universal sulfonate donor, various sulfotransferases of different types were screened to judge whether the APS might be utilized. The chondroitin 4-O-sulfotransferase (C4ST, amino acid sequence set forth in SEQ ID NO: 14), a heparin N-sulfotransferase (NST, amino acid sequence set forth in SEQ ID NO: 15), a trehalose sulfotransferase (Gene ID: 66738808, Stf0, amino acid sequence set forth in SEQ ID NO: 13), or an estrogen sulfotransferase (Gene ID: 360268, EST, amino acid sequence set forth in SEQ ID NO: 16) were respectively selected. The above sulfotransferases reacted in a Tris-HCl reaction system. The reaction system included: 50 mM Tris-HCl with a pH value of 7.5, 10 mM APS, 10 mM substrate to be sulfated, and 0.5 g/L corresponding sulfotransferase. The reaction temperature was 35° C., and the reaction was performed for 12 h. A catalysis result was detected by using a liquid chromatography-mass spectrometry technology. The result is shown in FIG. 10. The substrate deacetylated heparin, the estrogen, the chondroitin sulfate, and the trehalose all generate sulfonate groups in corresponding positions by using the APS as the sulfonate donor, the N-sulfated heparin, estrogen sulfonate, chondroitin sulfate A, and trehalose-2-sulfate are generated, and it proves that the APS is a general active sulfonate donor.

### Example 12: Construction of APS Synthesis Catalysis System

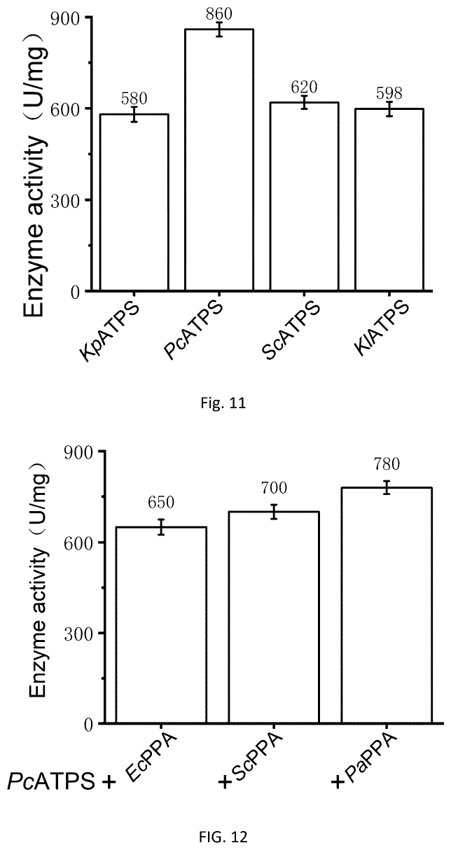

The APS was synthesized by catalyzing a substrate ATP and sulfate ions by an ATP sulfurylase, and pyrophosphate as a byproduct was generated. The ATP sulfurylases from different sources, including S. cerevisiae (Gene ID: 853466, ScATPS, amino acid sequence set forth in SEQ ID NO:1), Kluyveromyces lactis (Gene ID: 2894185, KIATPS, amino acid sequence set forth in SEQ ID NO:3), P. pastoris (Gene ID: 8196926, KpATP, amino acid sequence set forth in SEQ ID NO: 2), and Penicillium chrysogenum (Protein ID: CAP86100.1, PcATPS, amino acid sequence set forth in SEQ ID NO: 4), were compared and screened. The screened ATP sulfurylases were expressed and purified, and then, the specific enzyme activity was measured. As shown in FIG. 11, the activity of the ATP sulfurylase PcATPS from the source of P. chrysogenum is 860 U/mg to a maximum degree, the enzyme activity of ScATPS is 620 U/mg, the enzyme activity of KIATPS is 598 U/mg, and the enzyme activity of KpATPS is 580 U/mg.

In order to further improve the APS conversion efficiency, the pyrophosphatase was introduced into the APS synthesis system. The pyrophosphatase hydrolyzes the pyrophosphate to pull the reaction to proceed in a forward direction, and the catalysis efficiency of the ATP sulfurylase was improved. The ATP sulfurylases from different sources, including E. coli (Gene ID: 948748, EcPPA, amino acid sequence set forth in SEQ ID NO: 5), P. aeruginosa (Gene ID: 879025, PaPPA, amino acid sequence set forth in SEQ ID NO: 6), and S. cerevisiae (Gene ID: 855309, ScPPA, amino acid sequence set forth in SEQ ID NO: 7) were compared and screened. The proteins were added into the APS synthesis system after expression and purification, and the promotion effect on the ATP sulfurylase was respectively determined. The result is shown in FIG. 12. Through the introduction of the pyrophosphatase, the activity of the ATP sulfurylase can be respectively improved. The promotion effect of the P. aeruginosa on the ATP sulfurylase is most obvious.

### Example 13: Screening of Enzyme for Converting AMP into ATP

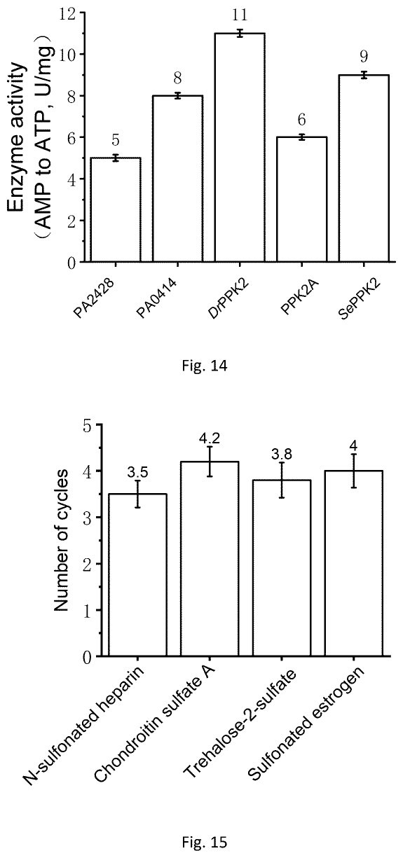

In order to realize the APS regeneration, the AMP needed to be converted into ATP again. Therefore, the polyphosphate kinases from different sources were screened. Polyphosphate kinases from the sources of P. aeruginosa (PA2428, Gene ID: 882843, amino acid sequence set forth in SEQ ID NO: 8; PA0414, Gene ID: 879494, amino acid sequence set forth in SEQ ID NO: 9), C. glutamicum (PPK2A, Protein ID: WP_011013972.1, amino acid sequence set forth in SEQ ID NO: 10), S. epidermidis (SePPK2, CDD: 274736, amino acid sequence set forth in SEQ ID NO: 11), and D. radiodurans (DrPPK2, CDD: 274737, amino acid sequence set forth in SEQ ID NO: 12) were respectively selected. Gene segments encoding the polyphosphate kinase were synthesized, and were ligated into the plasmid pET28a (+) between Nde I and Hind III restriction sites to obtain recombinant plasmids. The recombinant plasmids were transferred into E. coli BL21 (DE3) cells, and were cultured at 30° C. for 2 h. Then, induction was continuously performed for 10 h after 0.5 mM IPTG was added. Intracellular enzymes were collected, and the enzyme activity was measured. The result showed that the polyphosphate kinase had the activities of the AMP phosphorylase and the ADP phosphorylase at the same time, and might be used for one-step direct synthesis of ATP from AMP by using polyP_{n>2} as a phosphate donor. As shown in FIG. 14, when the AMP is used as a substrate, polyP_{n>2} is added, the generation of ATP can be detected through the detection on the product. It shows that the above polyphosphate kinase can be used for directly converting AMP into ATP. The DrPPK2 enzyme activity can reach 11 U/mg, and the enzyme activity of the polyphosphate kinases from other sources can be respectively as follows: PA2428: 5 U/mg; PA0414: 8 U/mg; PPK2A: 6 U/mg; and SePPK2: 9 U/mg.

### Example 14: Construction of APS Circulation Regeneration System

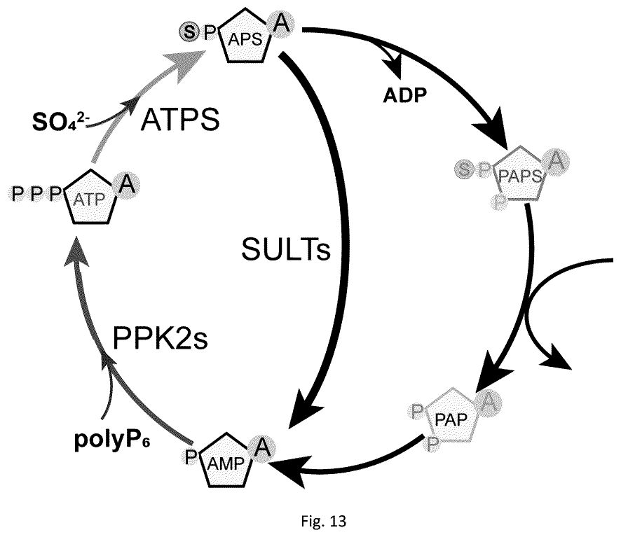

As shown in FIG. 13, an APS circulation regeneration system was constructed. By using the APS circulation regeneration system, under the catalysis effect of the enzyme, the APS might be catalyzed to synthesize the AMP, the AMP was catalyzed to synthesize ATP, and the ATP was catalyzed to synthesize APS, so that a closed-loop circulation catalytic reaction process was formed.

The APS was used for synthesizing the AMP under the catalysis of the sulfotransferase. The sulfotransferase included but was not limited to a chondroitin 4-O-sulfotransferase (C4ST, amino acid sequence set forth in SEQ ID NO: 14), a heparin N-sulfotransferase (NST, amino acid sequence set forth in SEQ ID NO: 15), a trehalose sulfotransferase (amino acid sequence set forth in SEQ ID NO: 13), and an estrogen sulfotransferase (amino acid sequence set forth in SEQ ID NO: 16). The AMP was used for generating the ATP under the participation of polyphosphate and the catalysis effect of the polyphosphate kinase. The polyphosphate kinase might be the polyphosphate kinase with the amino acid sequence set forth in SEQ ID NO: 8 to SEQ ID NO: 12. The ATP was used for generating the APS under the participation of sulfate and the catalysis effect of the ATP sulfurylase. The ATP sulfurylase might be the ATP sulfurylase with the amino acid sequence set forth in any one of SEQ ID NO: 1 to SEQ ID NO: 4.

In the APS circulation regeneration system, a catalysis system containing an enzyme preparation might participate in a reaction. The catalysis system was as follows: during reaction in a Tris-HCl reaction system, the reaction system included 50 mM Tris-HCl having a pH value of 7.5, 10 mM ATP, 20 mM MgSO₄, 80 mM K₂SO₄, 20 mM polyP₆, 10 mM substrate to be sulfated, 0.5 g/L corresponding sulfotransferase, and 0.5 g/L ATP sulfurylase. The reaction temperature was 35° C., and the reaction was performed for 12 h. The above enzyme might be respectively expressed in common microbial hosts such as E. coli, Bacillus subtilis, P. pastoris, and S. cerevisiae, and might be used in a cell form. The enzyme expressed by the microbial cells might be collected, and was subjected to affinity chromatography purification to obtain a pure enzyme. Then, after quantification, the pure enzyme was put into 50 mM Tris-HCl to prepare a mixed enzyme preparation. A substrate (polyP₆ or MgSO₄) might be added in use.

### Example 15: Application of APS Regeneration System to Production of Chondroitin Sulfate

The APS regeneration system included: 0.5 g/L polyphosphate kinase SePPK2, 0.5 g/L ATP sulfurylase ScATPS, 20 mM polyP₆, 10 mM ATP, 20 mM MgSO₄, and 80 mM K₂SO₄. The APS regeneration system was applied to production of the chondroitin sulfate A. That was, 0.5 g/L C4ST pure enzyme and 5 g/L chondroitin precursor metered by the final concentration were added into the system of the APS regeneration system. The reaction was performed at 35° C. for 6 h. An experiment catalysis group in the absence of 0.5 g/L PPK2 and 20 mM polyP₆ was used as a control. The product chondroitin sulfate was subjected to analysis and quantification, and the circulation capability of the APS regeneration system in the chondroitin sulfate production was calculated. The result is shown in FIG. 15.

### Example 16: Application of APS Regeneration System to Production of Heparin

The APS regeneration system included: 0.5 g/L polyphosphate kinase SePPK2, 0.5 g/L ATP sulfurylase ScATPS, 20 mM polyP₆, 10 mM ATP, 20 mM MgSO₄, and 80 mM K₂SO₄, and was applied to production of heparin. That was, 0.5 g/L NST pure enzyme and 5 g/L N-desulfated heparin precursors were added into the APS regeneration system. Reaction was performed at 35° C. for 6 h. An experiment catalysis group in the absence of 0.5 g/L PPK2 and 20 mM polyP₆ was used as a control. The product N heparin sulfonate was subjected to analysis and quantification, and the circulation capability of the APS regeneration system in the heparin production was calculated. The result is shown in FIG. 15.

### Example 17: Application of APS Regeneration System to Production of Trehalose-2-Sulfonate

The APS regeneration system included: 0.5 g/L polyphosphate kinase SePPK2, 0.5 g/L ATP sulfurylase ScATPS, 20 mM polyP₆, 10 mM ATP, 20 mM MgSO₄, and 80 mM K₂SO₄, and was applied to production of trehalose-2-sulfonate. That was, 0.5 g/L Stf0 pure enzyme and 5 g/L trehalose precursors were added into the APS regeneration system. After sufficient reaction, reaction was performed at 35° C. for 6 h, and an experiment catalysis group in the absence of 0.5 g/L PPK2 and 20 mM polyP₆ was used as a control. The product of trehalose-2-sulfonate was subjected to analysis and quantification, and the circulation capability of the APS regeneration system in the trehalose-2-sulfonate production was calculated. The result is shown in FIG. 15.

### Example 18: APS Binding Domain 3′PB Re-Edition for Improving Circulation Capability of APS Regeneration System

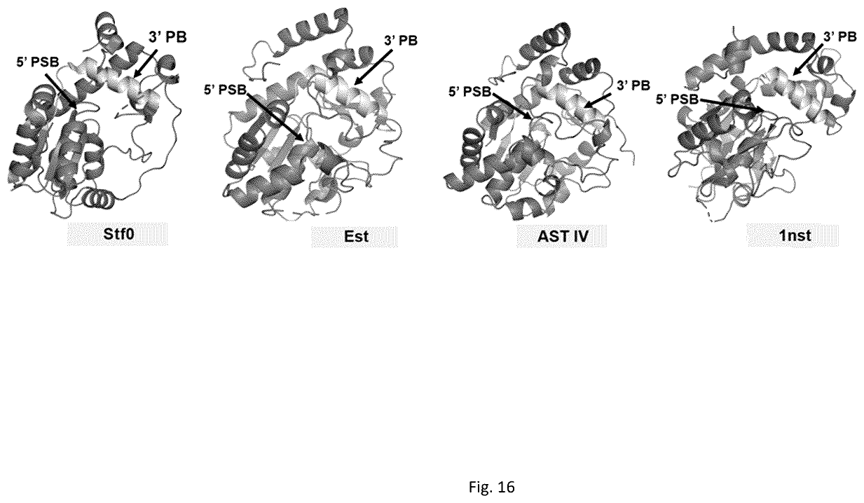

By comparing the binding region 3′PB motifs of different sulfotransferases that bind the APS (FIG. 16), the binding of the 3′PB regions on the APS and the PAPS was very important. The 3′PB motif was a region for binding 3′OH in the APS structure. The engineering of the 3′PB motif region was very important for improving the affinity of the sulfotransferases for the APS.

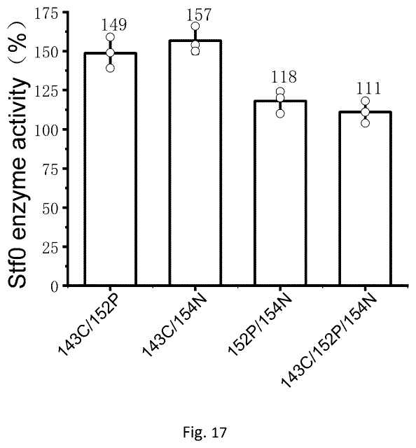

By taking the trehalose sulfotransferase (SEQ ID NO: 13) as an example, the positions of amino acids directly acting with the APS were determined to be positions 143, 152 and 154 through molecular docking. Saturation mutagenesis was respectively performed on them: the 143-position arginine was determined to be mutated into cysteine, the 152-position serine was determined to be mutated into proline, and the 154-position tryptophan was determined to be mutated into asparaginate. A single mutant was constructed. Superimposed mutation was performed based on single mutation. During mutation enzyme expression, a single colony was picked to be cultured in an LB Culture over the night. After the transfer and inoculation into a fermentation culture medium of a TB Culture for culture at 37° C. for 2 h at an inoculation amount of 1% (v/v), 0.5 mM IPTG was added, and the material was transferred to a 30° C. environment to be continuously cultured for 12 h. The specific enzyme activities of a wild enzyme and the mutation enzyme were determined. The result showed that the specific enzyme activity of the wild enzyme was 3.2 U/mg protein, and the mutant greatly improved the APS utilization compared with the wild enzyme (FIG. 17). When the mutant was applied to the constructed APS regeneration system, its sulfonation efficiency was improved by two times compared with that of the wild type. References might be provided for the mutation of other sulfotransferases.

### Comparative Example 1

The specific embodiment was the same as Example 11. The difference was that the pyrophosphatase in Example 11 was replaced with the same amount of PCATPS (Protein ID: CAP86100.1). The result showed that the APS generation speed was not accelerated.

### Comparative Example 2

The specific embodiment was the same as Example 17. The difference was that a mutant mutating the 142-position substance to cysteine was also constructed. The result showed that the mutation at this site had no improvement effect on the APS affinity.

Although the exemplary examples of the present disclosure have been provided above, they are not intended to limit the present disclosure. Those skilled in the art will appreciate that various changes and modifications might be made without departing from the spirit and scope of the present disclosure. Therefore, the protection scope of the present disclosure shall be defined by the claims.

What is claimed is:

1. A 3′-phosphoadenosine-5′-phosphosulfate (PAPS) regeneration circulation system, comprising a polyphosphate kinase, a 3′-adenosine phosphate-5′-phosphate (PAP) dephosphorylation enzyme, and a PAPS synthesis bifunctional enzyme which is a combination of an ATP sulfurylase and an APS kinase, being applicable to PAPS synthesis through catalysis by one or more of the above enzymes from any one substance of AMP, ADP, ATP, APS, or PAP and wherein the PAPS regeneration circulation system comprises catalyzing module I, module II, and module III forming a closed-loop cycle, wherein the catalyzing module I converts the AMP into the ATP, the catalyzing module II converts the ATP into the PAPS, and the catalyzing module III converts the PAPS into the AMP.

2. The PAPS regeneration circulation system according to claim 1, wherein the polyphosphate kinase has ADP and AMP phosphorylation bifunctional activity.

3. The PAPS regeneration circulation system according to claim 2, using cells as carriers.

4. The PAPS regeneration circulation system according to claim 1, further comprising a sulfotransferase, polyphosphate polyP_{n>2}, and sulfate.

5. A method for preparing a sulfonation product, comprising: taking a catalytic reaction in the PAPS regeneration circulation system according to claim 1, wherein the sulfonation product comprises synthesis of heparin, chondroitin sulfate, hirudin, or flavone.

6. The method according to claim 5, comprising: adding a sulfotransferase into a reaction system comprising the PAPS regeneration circulation system to produce the sulfonation product, wherein the sulfotransferase comprises a chondroitin 4-O-sulfotransferase, a heparin N-sulfotransferase, a trehalose sulfotransferase, or an estrogen sulfotransferase.


SEQUENCE LISTING (30 sequences)

SEQ ID NO: 1 - protein, 511 aa, synthetic construct
MPAPHGGILQDLIARDALKKNELLSEAQSSDILVWNLTPRQLCDIELILNGGFSPLTGFLNENDYSSVVTDSRLADGTLWTIPITLDVDEAFANQIKPDTRIALFQDDEIPIAILTVQDVYKPNKTIEAEKVFRGDPEHPAISYLFNVAGDYYVGGSLEAIQLPQHYDYPGLRKTPAQLRLEFQSRQWDRVVAFQTRNPMHRAHRELTVRAAREANAKVLIHPVVGLTKPGDIDHHTRVRVYQEIIKRYPNGIAFLSLLPLAMRMSGDREAVWHAIIRKNYGASHFIVGRDHAGPGKNSKGVDFYGPYDAQELVESYKHELDIEVVPFRMVTYLPDEDRYAPIDQIDTTKTRTLNISGTELRRRLRVGGEIPEWFSYPEVVKILRESNPPRPKQGFSIVLGNSLTVSREQLSIALLSTFLQFGGGRYYKIFEHNNKTELLSLIQDFIGSGSGLIIPNQWEDDKDSVVGKQNVYLLDTSSSADIQLESADEPISHIVQKVVLFLEDNGFFVF

SEQ ID NO: 2 - protein, 547 aa, synthetic construct
MPSPHGGVLQDLIKRDASIKEDLLKEVPQLQSIVLTGRQLCDLELILNGGFSPLTGFLTEKDYRSVVDDLRLASGDVWSIPITLDVSKTEASKFRVGERVVLRDLRNDNALSILTIEDIYEPDKNVEAKKVFRGDPEHPAVKYLFDVAGDVYIGGALQALQLPTHYDYTALRKTPAQLRSEFESRNWDRVVAFQTRNPMHRAHRELTVRAARANLANVLIHPVVGLTKPGDIDHHTRVKVYQEIIKKYPNGMAQLSLLPLAMRMAGDREAVWHAIIRKNYGASHFIVGRDHAGPGKNSAGVDFYGPYDAQELVEKYKDELDIQVVPFRMVTYLPDEDRYAPIDTVKEGTRTLNISGTELRKRLRDGTHIPEWFSYPEVVKILRESNPPRPKQGFTLYLTGLPNSGVDALSNALVATFNQFEGARHITLLDGKNVNESALPFVAHELTRSGAGVIIADPTKAPSAAEIDSIRKEVSKAGSFIVISLTTPLNQVSQHDRKGYYSTSRKDVDNYVFPEDAEIKIDLAKEGAIVGIQKVVLYLEEQGFFQF

SEQ ID NO: 3 - protein, 502 aa, synthetic construct
MPSPHGGVLQDLVARDASKKAELLEIAQSGDLTSWSLTARQICDLELILNGGFSPLDGFLNQQDYQSVVEKSRLQNGLVWTIPITLDVDAEFASQLSPDQRIVLLQDNEFPLAILTVSDVYQPDKAVEAKKVFRGDPEHPAVKYLFEQAGEFYVGGSLEAIQLPVHYDYPGWRKTPAQLRLEFESKQWDRVVAFQTRNPMHRAHRELTVRAARSNNSKILIHPVVGLTKPGDIDHHTRVRVYQEIIKRYPNGMAQLSLLPLAMRMGGDREAVWHAIIRKNYGASHFIVGRDHAGPGKNSKGVDFYGPYDAQELVESYKNELDIEVVPFRMVTYLPDEDRYAPIDEIDTDKTRTLNISGTELRNRLRDGGEIPAWFSYPEVVKILRESNPSRPKQGFALVLSETLPAQLKTALLSTFLQYGGGRHYKVLEHGNNEEILALVPDFVRSGTGLILQNASSLKGTNVFKIGEESGSDIPLETEDKNILHIVQRVVLFLEDQGFFQF

SEQ ID NO: 4 - protein, 573 aa, synthetic construct
MANAPHGGVLKDLLARDAPRQAELAAEAESLPAVTLTERQLCDLELIMNGGFSPLEGFMNQADYDRVCEDNRLADGNVFSMPITLDASQEVIDEKKLQAGSRITLRDFRDDRNLAILTIDDIYRPDKTKEAKLVFGGDPEHPAIVYLNNTVKEFYIGGKIEAVNKLNHYDYVALRYTPAELRVHFDKLGWSRVVAFQTRNPMHRAHRELTVRAARSRQANVLIHPVVGLTKPGDIDHFTRVRAYQALLPRYPNGMAVLGLLGLAMRMGGPREAIWHAIIRKNHGATHFIVGRDHAGPGSNSKGEDFYGPYDAQHAVEKYKDELGIEVVEFQMVTYLPDTDEYRPVDQVPAGVKTLNISGTELRRRLRSGAHIPEWFSYPEVVKILRESNPPRATQGFTIFLTGYMNSGKDAIARALQVTLNQQGGRSVSLLLGDTVRHELSSELGFTREDRHTNIQRIAFVATELTRAGAAVIAAPIAPYEESRKFARDAVSQAGSFFLVHVATPLEHCEQSDKRGIYAAARRGEIKGFTGVDDPYETPEKADLVVDFSKQSVRSIVHEIILVLESQGFLERQ

SEQ ID NO: 5 - protein, 176 aa, synthetic construct
MSLLNVPAGKDLPEDIYVVIEIPANADPIKYEIDKESGALFVDRFMSTAMFYPCNYGYINHTLSLDGDPVDVLVPTPYPLQPGSVIRCRPVGVLKMTDEAGEDAKLVAVPHSKLSKEYDHIKDVNDLPELLKAQIAHFFEHYKDLEKGKWVKVEGWENAEAAKAEIVASFERAKNK

SEQ ID NO: 6 - protein, 175 aa, synthetic construct
MSYSKIPAGKDLPNDIYVAIEIPANHAPIKYEIDKDTDCLFVDRFMATPMFYPANYGFIPNTLADDGDPLDVLVVTPYPVAPGSVIRARPVGVLHMTDEAGGDAKLIAVPHDKLSVLYKDVKEYTDLPALLLEQIKHFFENYKDLEKGKWVKVEGWGNADAARAEITKAVAAFQK

SEQ ID NO: 7 - protein, 310 aa, synthetic construct
MNLLRMNALTSKARSIERLKQTLNILSIRNHRQFSTIQQGSKYTLGFKKYLTLLNGEVGSFFHDVPLDLNEHEKTVNMIVEVPRWTTGKFEISKELRFNPIVQDTKNGKLRFVNNIFPYHGYIHNYGAIPQTWEDPTIEHKLGKCDVALKGDNDPLDCCEIGSDVLEMGSIKKVKVLGSLALIDDGELDWKVIVIDVNDPLSSKIDDLEKIEEYFPGILDTTREWFRKYKVPAGKPLNSFAFHEQYQNSNKTIQTIKKCHNSWKNLISGSLQEKYDNLPNTERAGNGVTLEDSVKPPSQIPPEVQKWYYV

SEQ ID NO: 8 - protein, 325 aa, synthetic construct
LVPRGSHMASMTGGQQMGRGSMDSYGDTSGRIGRDWLDRHDEELEQELLDDELNLDELFGPEQEDAPGELSRRRYFRELFRLQRELVKLQNWVVHTGHKVVILFEGRDAAGKGGVIKRITQRLNPRVCRVAALPAPNDREQTQWYFQRYVSHLPAGGEIVLFDRSWYNRAGVERVMGFCNDEQYEEFFRSVPEFEKMLARSGIQLLKYWFSISDAEQHLRFLSRIHDPLKQWKLSPMDLESRRRWEAYTKAKETMLERTHIPEAPWWVVQADDKKRARLNCIHHLLQQMPYREVPQPPVHLPERLRHADYVRHPTPGEIIVPEVY

SEQ ID NO: 9 - protein, 298 aa, synthetic construct
MALQVASAPHGSSEDSTSASLPANYPYHTRMRRNEYEKAKHDLQIELLKVQSWVKETGQRVVVLFEGRDAAGKGGTIKRFMEHLNPRGARIVALEKPSSQEQGQWYFQRYIQHLPTAGEMVFFDRSWYNRAGVERVMGFCSPLQYLEFMRQAPELERMLTNSGILLFKYWFSVSREEQLRRFISRRDDPLKHWKLSPIDIKSLDKWDDYTAAKQAMFFHTDTADAPWTVIKSDDKKRARLNCIRHFLHSLDYPDKDRRIAHEPDPLLVGPASRVIEEDEKVYAEAAAAPGHANLDIPA

SEQ ID NO: 10 - protein, 320 aa, synthetic construct
MRKKKDGQNLPDFRKNPPKLDKKAYEKELKRLQAELVDLQQWVVETGARVVIVMEGRDAAGKGSAIKRITQYLNPRSARIEALPTPNSREKGQWYFQRYIEKLPTAGEIVIFDRSWYNRAGVERVMGFCTSQEYRRFLHQAPIFERLLVEDGIHLRKYWFSVSDEEQIERFEDRLSDPLRRWKLSPMDLQSITRWEDYSRAKDEMFIHTDIPSAPWYTVESEDKKRSRINVISHLLSTIPYEKIDRPLPEIPHRPDSESDYVRPPRDEFRYVPDVAAHLEEERIKKEEKAKKAKKPAKAAGKNSDKQKSSGGKGKKKSKK

SEQ ID NO: 11 - protein, 464 aa, synthetic construct
LEELQLKVARLSRRTHELGIPIMVLFEGIPASGKTRLSNELLLHLDAKYSRFIATKSPESNDLRYQFLQKYWNTLPQKGNINIYFRSWYSHFLDYKENKIKHDQYKNYDVLVNQIYHFESMLKNDNYEIIKFFIEINEEKRNEHIQQTKDNPLTRWKVQEYENVIPQESYLNQMHQFINKDKDWKVIDYTEREHAFEKMYLHLIDRLEQAIKKVEQQTTKVNGKFTSSFTTSLFNNNLEKVDKKTYKNLIVELQQRMREIQFALYERKIPLVLVFEGMDAAGKGGNIKRIREKLDPTGYEVNGISAPTDVELKHHYLWRFAKKMPKSGHIEIFDRSWYGRVLVERVEGFASQNEWQRASDEINQFEKMWTDEGTIILKFFLCLDKDEQLKRFKDRENNPDKQWKITEEDWRNREKWDKYLEASHDMIESTNTSYAPWYIVLADHKKTSRIEVLKTIIRKCEEVL

SEQ ID NO: 12 - protein, 261 aa, synthetic construct
IDNYRVKPGKRVKLSDWATNDDAGLSKEEGQAQTAKLAGELAEWQERLYAEGKQSLLLILQARDAAGKDGAVKKVIGAFNPAGVQITSFKQPSAEELSHDFLWRIHQKAPAKGYVGVFNRSQYEDVLVTRVYDMIDDKTAKRRLEHIRHFEELLTDNATRIVKVYLHISPEEQKERLQARLDNPGKHWKFNPGDLKDRSNWDKFNDVYEDALTTSTDDAPWYVVPADRKWYRDLVLSHILLGALKDMNPQFPAIDYDPSKV

SEQ ID NO: 13 - protein, 267 aa, synthetic construct
MSDHPTAYLVLASQRSGSTLLVESLRATGVAGEPQEFFQYLPNTSMSPQPREWFADVEDQSILRLLDPLIEGKPDLAPATIWRDYIQTVGRTPNGVWGGKLMWNQTPLLVQRAKDLPDRSGSGLLSAIRDVVGSDPVLIHIHRPDVVSQAVSFWRAVQTRVWRGRPDPVRDARAEYHAGAIAHVITMLRAQEEGWRAWFTEENVEPIDVDYPYLWRNLTEVVGTVLEALGQDPRLAPKPVLERQADQRSDEWVERYRRDAQRDGLPL

SEQ ID NO: 14 - protein, 460 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADIGSSPLQELYNPIQLELSNTAILHQMRRDQVTDTCRANSAMSRKRRVLTPNDLKHLVVDEDHELIYCYVPKVACTNWKRLMMVLSGRGKYSDPMEIPANEAHVSANLKTLNQYSIPEINHRLKSYMKFLFVREPFERLVSAYRNKFTQKYNTSFHKRYGTKIIRRQRKNATQEALRKGDDVKFEEFVAYLIDPHTQREEPFNEHWQTVYSLCHPCHIHYDLVGKYETLEEDSNYVLQLAGVSGYLKFPTYAKSTRTTDEMTTEFFQNISAEHQTQLYEVYKLDFLMFNYSVPNYLKLD

SEQ ID NO: 15 - protein, 882 aa, synthetic construct [sequence omitted]

SEQ ID NO: 16 - protein, 295 aa, synthetic construct
METSMPEYYEVFGEFRGVLMDKRFTKYWEDVEMFLARPDDLVIATYPKSGTTWISEVVYMIYKEGDVEKCKEDAIFNRIPYLECRNEDLINGIKQLKEKESPRIVKTHLPPKLLPASFWEKNCKMIYLCRNAKDVAVSYYYFLLMITSYPNPKSFSEFVEKFMQGQVPYGSWYDHVKAWWEKSKNSRVLFMFYEDMKEDIRREVVKLIEFLERKPSAELVDRIIQHTSFQEMKNNPSTNYTMMPEEMMNQKVSPFMRKGIIGDWKNHFPEALRERFDEHYKQQMKDCTVKFRMEL

SEQ ID NO: 17 - DNA, 1395 bp, synthetic construct
  source 1..1395: mol_type = other DNA
ctggaagaactgcagctgaaagtggcgcgcctgagccgccgcacccatgaactgggcattccgattatggtgctgttcgaaggcattccggccagcggcaaaacccgcctgagcaacgaactgctgctgcatctggatgcgaaatacagccgcttcattgcgaccaaaagcccggaaagcaacgatctgcgttaccagttcctgcagaaatactggaacaccctgccgcagaaaggcaatattaacatttattttcgcagctggtacagccattttctggattataaagaaaataaaattaaacatgatcagtataaaaattatgatgtgctggtgaaccagatttatcattttgaaagcatgctgaaaaacgacaactatgaaatcattaaattttttattgaaattaatgaagaaaaacgcaacgaacatatccagcagacgaaagataatccgctgacccgttggaaagtgcaggaatatgaaaatgttattccgcaggaaagctatctgaaccagatgcatcagtttattaacaaagataaagattggaaagtgattgattataccgaacgcgaacatgcgttcgaaaaaatgtatctgcatctgattgatcgcctggaacaggcgattaaaaaagtggagcagcagaccaccaaagtgaatggcaaatttacctcctcgttcacgaccagcctgtttaacaacaatctggagaaagtcgataaaaaaacctataaaaatctgattgtcgaactgcagcagcgtatgcgcgaaattcagtttgccctgtacgaacgcaaaattccgctggtactggtgtttgaaggcatggatgcggcgggcaaaggtggcaacatcaaacgcatccgtgaaaaactggatccgaccggctacgaagttaatggcattagcgcgccgaccgatgtagaactgaaacaccactatctgtggcgttttgcgaaaaaaatgccgaaatcgggtcacattgaaatttttgatcgtagctggtacggccgcgtgctggtggaacgcgtggaaggctttgcgagccagaatgaatggcagcgcgccagcgatgaaattaaccagttcgaaaaaatgtggaccgatgaaggcaccatcattctgaaattcttcctgtgcctggataaagatgaacagctgaaacgcttcaaagatcgcgaaaataacccggataaacagtggaaaattaccgaagaggattggcgcaatcgcgaaaaatgggataaatatctggaagcgagccatgatatgattgaaagcaccaataccagctatgcgccgtggtatattgtgctggcggatcataaaaaaacctctcgcattgaagtgctgaaaaccattatccgcaaatgtgaagaggtgctgtaa

SEQ ID NO: 18 - DNA, 786 bp, synthetic construct
  source 1..786: mol_type = other DNA
attgataactatcgcgtgaaaccgggcaaacgcgtgaaactgagtgattgggccaccaacgatgatgcgggcctgagcaaagaagaaggccaggcccagaccgcgaaactggcgggcgaactggcggaatggcaggaacgcctgtatgcggaaggcaaacagagcctgctgctgattctgcaggcgcgtgatgcggcgggcaaagatggcgcggtgaaaaaagttattggcgcgttcaatccggcgggcgtgcagattaccagctttaaacagccgagcgcggaagaactgagccacgatttcctgtggcgcattcatcagaaagcgccggcgaaaggttacgtgggtgtatttaaccgcagccagtatgaagatgttctggtgacccgtgtgtacgatatgattgatgataaaaccgccaaacgtcgtctggaacacatccgccattttgaagaactgctgaccgataatgcgacccgtattgtgaaagtgtacctgcatatcagcccggaagaacagaaagaacgcctgcaggcgcgcctggataaccccggcaaacactggaaattcaatccgggtgatctgaaagatcgcagtaactgggataaatttaacgatgtgtatgaagatgcgctgaccaccagcaccgatgatgcgccgtggtacgtggtgccggcagatcgtaaatggtaccgtgatctggtgctgagccatattctgctgggcgccctgaaagatatgaacccgcagttcccggcgattgattacgatccgagcaaagtgtga

SEQ ID NO: 19 - DNA, 801 bp, synthetic construct
  source 1..801: mol_type = other DNA
atgtccgatcaccctactgcgtacctggttctggcttctcagcgttctggctctactctgctggtagaatctctgcgtgctaccggtgttgcgggcgaaccgcaagaattcttccagtacctgcctaacacctctatgtctccgcagccgcgtgagtggtttgctgacgtggaagaccagagcatcctgcgtctgctggacccgctgatcgaaggtaaaccggacctggcgccagcgactatttggcgtgattacattcagaccgtgggtcgtactccgaacggcgtatggggtggcaaactgatgtggaaccagaccccgctgctggtacagcgtgcgaaagacctgcctgatcgttctggttccggtctgctgtccgctattcgcgacgttgttggctccgatccggttctgattcacatccaccgtccggacgttgtttcccaggcagtttccttctggcgcgctgttcagacccgtgtatggcgcggtcgccctgatccggtacgtgatgctcgtgctgagtaccatgcaggtgctatcgcgcacgttatcactatgctgcgcgctcaggaagaaggttggcgtgcgtggttcaccgaggagaacgtggaaccgattgatgttgactatccatacctgtggcgcaacctgaccgaagtagttggtaccgtgctggaagccctgggccaggaccctcgtctggctcctaagcctgtactggaacgccaggcagaccaacgtagcgatgaatgggtagagcgttatcgtcgtgatgcccagcgtgacggcctgccgctg

SEQ ID NO: 20 - DNA, 2646 bp, synthetic construct
  source 1..2646: mol_type = other DNA
atgcctgccctggcctgtctgcgccgtctgtgccgccacgtctctccgcaggctgtgctgttcctgctgtttatcttttgtctgttctccgtgttcatttctgcgtactacctgtacggctggaaacgcggtctggaaccgagcgcagatgcaccagaacctgactgcggtgatcctgctccggttgctccgtcccgtctgctgccgctgaaaccagtccaggcggctaccccgagccgcaccgatccgctggtcctggtattcgtcgagtccctgtattctcagctgggccaagaggttgtcgcgattctggagtcttctcgcttcaaataccgtactgaaatcgcgccgggcaaaggtgatatgccgactctgactgataaaggccgtggccgcttcgctctgatcatctacgaaaacatcctgaagtacgtgaatctggatgcttggaaccgcgaactgctggataaatactgtgttgcgtacggcgtgggtatcatcggttttttcaaagctaacgaaaactccctgctgagcgcacagctgaagggtttcccgctgttcctgcactccaatctgggtctgaaggattgtagcattaacccgaaatctccgctgctgtacgtgacccgtccaagcgaggttgagaaaggcgttctgccgggcgaagactggactgtgttccagtctaaccattccacctatgaaccggtcctgctggccaaaactcgctctagcgagagcatcccacatctgggcgcggacgcgggtctgcatgcagcgctgcatgcgaccgttgttcaagatctgggtctgcacgacggtattcagcgtgttctgttcggtaacaatctgaatttttggctgcacaaactggtgttcgttgacgctgtagcatttctgactggtaagcgtctgtctctgccgctggaccgttatatcctggtagacatcgacgacatctttgtaggtaaggaaggcacccgtatgaaagtcgaggacgttaaagcgctgtttgacacccaaaacgagctgcgcgctcatattccgaatttcaccttcaacctgggttacagcggtaagttcttccacactggcactaacgcggaggacgcaggtgacgatctgctgctgtcttatgttaaagaattctggtggttcccgcatatgtggtcccacatgcagcctcacctgttccacaaccagtctgtactggccgaacagatggcgctgaacaaaaaatttgcagttgaacatggtatcccgaccgacatgggctacgcggtagcaccacaccactctggtgtttacccagtacacgttcagctgtacgaggcgtggaaacaggtgtggtctattcgcgtgacgtccaccgaagaatacccgcatctgaaaccagctcgttatcgccgtggcttcattcataacggtatcatggttctgcctcgccagacgtgtggtctgtttactcacactattttctacaacgaatacccgggtggttctagcgaactggacaaaatcattaacggtggtgaactgttcctgaccgttctgctgaacccgatcagcatcttcatgacgcacctgtctaattacggcaacgatcgtctgggtctgtacacgtttaaacacctggttcgtttcctgcactcttggactaacctgcgcctgcagaccctgccaccggtgcagctggctcagaaatactttcagatcttctccgaagagaaagacccgctgtggcaggacccgtgcgaagacaaacgccacaaagacatctggagcaaggaaaaaacctgcgaccgtttcccgaaactgctgatcatcggtccgcagaaaacgggtactaccgcgctgtatctgttcctgggtatgcacccggacctgtcttccaactacccttcttctgaaacctttgaagaaatccagttctttaacggccacaactatcataaaggcatcgactggtacatggagttcttcccgatcccgtctaacaccaccagcgacttttactttgaaaaaagcgcgaactatttcgacagcgaagttgcgccgcgtggcgctgcagcactgctgccgaaagccaaagtgctgaccatcctgatcaacccagcggaccgtgcttattcctggtaccagcaccaacgtgcgcacgatgatccggttgctctgaaatacaccttccatgaagttatcaccgcaggctctgatgcttcttctcgtctgcgtgccctgcagaatcgctgtctggttcctggttggtacgcgactcatatcgaacgttggctgtctgcgtaccacgcaaaccagatcctggtgctggacggcaagctgctgcgtaccgaaccggcaaaagttatggacatggtccagaaattcctgggtgtcaccaacactatcgactaccataaaactctggcgttcgatccgaaaaaaggtttttggtgccagctgctggaaggcggtaaaactaagtgcctgggtaaatctaaaggccgtaaatacccggaaatggatctggacagccgcgcgtttctgaaagattattaccgcgaccacaacatcgaactgtccaaactgctgtataaaatgggccagaccctgccgacttggctgcgtgaggatctgcagaacacgcgc

SEQ ID NO: 21 - DNA, 885 bp, synthetic construct
  source 1..885: mol_type = other DNA
atggagacgtcaatgccggaatactatgaagtattcggcgagttccgtggggttttgatggacaaacgcttcaccaagtattgggaagacgtcgaaatgttcttagctcgccctgacgacttagtgattgcgacttacccgaaatcagggactacgtggatttctgaagtcgtctatatgatctacaaggaaggagacgtcgaaaaatgtaaagaggatgctattttcaatcgcattccataccttgaatgccgcaacgaagacctgattaacgggatcaaacaattgaaggagaaagagagcccccgcatcgttaaaacacacctgccccctaagttgcttccggcgagtttttgggaaaaaaattgcaaaatgatctacttatgccgtaacgcgaaggatgtcgcagttagttactattatttccttctgatgattaccagctatcccaaccctaagtccttttcagagtttgtcgagaaattcatgcaggggcaggtaccgtatggtagttggtatgaccatgtaaaggcgtggtgggaaaagtcgaaaaactctcgtgtactgtttatgttttacgaggacatgaaagaagatatccgccgcgaggtcgtgaaactgattgagtttcttgagcgtaaaccatccgcggaactggtggaccgcatcattcagcatacctcgttccaggagatgaagaataatccgtccactaactatactatgatgccagaagaaatgatgaaccagaaggtatcgccgttcatgcgcaaaggcatcattggggattggaagaatcactttcctgaagcgttacgcgagcgctttgacgaacattacaaacagcaaatgaaggactgtactgtaaagtttcgtatggaactg

SEQ ID NO: 22 - protein, 246 aa, synthetic construct
MLDQVCQLARNAGDAIMQVYDGTKPMDVVSKADNSPVTAADIAAHTVIMDGLRTLTPDVPVLSEEDPPGWEVRQHWQRYWLVDPLDGTKEFIKRNGEFTVNIALIDHGKPILGVVYAPVMNVMYSAAEGKAWKEECGVRKQIQVRDARPPLVVISRSHADAELKEYLQQLGEHQTTSIGSSLKFCLVAEGQAQLYPRFGPTNIWDTAAGHAVAAAAGAHVHDWQGKPLDYTPRESFLNPGFRVSIY

SEQ ID NO: 23 - protein, 273 aa, synthetic construct
MRPVPWGELVALVRRAGEAILPHWRADVVVRSKADESPVTAADLAAHHILEAGLRALAPDIPVLSEEDCEIPLSERGHWRRWWLVDPLDGTKEFISGSEEFTVNVALVEDGRVLFGLVGVPVSGRCYYGGAGLGAWREEADGRAQPISVRLEPEEAFTVVASKRHGSPAQERLLDGLSERFGDLRRASIGSSLKFCLLAEGAADCYPRLTPTSQWDTAAAQGVLEGAGGEVLDLHGAPFTYEPREDYLNGSFLALPRAAEWRSELIQLARALH

SEQ ID NO: 24 - protein, 352 aa, synthetic construct
MFSREVKLAQLAVKRASLLTKRISDEIAARTVGGISKSDDSPVTVGDFAAQSIIINSIKKAFPNDEVVGEEDSAMLKKDPKLAEKVLEEIKWVQEQDKANNGSLSLLNSVDEVCDAIDGGSSEGGRQGRIWALDPIDGTKGFLRGDQFAVCLALIVDGVVKVGVIGCPNLPFDLQNKSKGKGGLFTAAEGVGSYYQNLFEEILPLESSKRITMNNSLSFDTCRVCEGVEKGHSSHGLQGLIKEKLQIKSKSANLDSQAKYCALSRGDAEIYLRLPKDVNYREKIWDHAAGNILIKESGGIVSDIYGNQLDFGNGRELNSQGIIAASKNLHSDIITAVKSIIGDRGQDLEKYI

SEQ ID NO: 25 - protein, 357 aa, synthetic construct
MALERELLVATQAVRKASLLTKRIQSEVISHKDSTTITKNDNSPVTTGDYAAQTIIINAIKSNFPDDKVVGEESSSGLSDAFVSGILNEIKANDEVYNKNYKKDDFLFTNDQFPLKSLEDVRQIIDFGNYEGGRKGRFWCLDPIDGTKGFLRGEQFAVCLALIVDGVVQLGCIGCPNLVLSSYGAQDLKGHESFGYIFRAVRGLGAFYSPSSDAESWTKIHVRHLKDTKDMITLEGVEKGHSSHDEQTAIKNKLNISKSLHLDSQAKYCLLALGLADVYLRLPIKLSYQEKIWDHAAGNVIVHEAGGIHTDAMEDVPLDFGNGRTLATKGVIASSGPRELHDLVVSTSCDVIQSRNA

SEQ ID NO: 26 - DNA, 915 bp, synthetic construct
  source 1..915: mol_type = other DNA
atggactcctatggcgatacctcgggccgcatcggtcgcgactggctcgatcgacacgacgaagagctggagcaggaactgctcgacgacgagctgaacctggacgagctgttcggtcccgagcaggaggatgcgcccggcgaattgagtcggcgccgctatttccgcgaactgttccgcctgcaacgggaactggtgaaactgcagaactgggtggtgcacaccgggcacaaggtggtgattctcttcgaggggcgcgacgcggcgggcaagggcggcgtgatcaagcgcatcacccagcgcctcaacccgcgggtctgccgggtcgccgcgctgcccgcgccgaacgaccgcgagcagacccagtggtatttccagcgctacgtctcgcacctgccggccggtggcgagatcgtcctgttcgaccgcagctggtacaaccgcgccggggtcgagcgggtcatgggtttctgcaacgacgaacaatacgaggaattcttccgcagcgtgcctgagttcgaaaagatgctggcgcgctcgggcatccagttgctcaagtactggttctccatctccgacgccgagcagcacctgcgtttcctcagccgtatccacgatccgctcaagcagtggaagctcagtccgatggacctggagtcgcggcggcgctgggaggcctacaccaaggccaaggaaaccatgctcgaacgcacccacatccccgaggcgccctggtgggtggtacaggccgacgacaagaagcgcgcgcggctgaactgcatccaccacctgttgcagcagatgccctaccgggaagttccgcagccgccggtacacctgccggagcgcctgcgccacgccgattacgtcaggcatccgacgcccggcgagatcatcgtgccggaagtctattaa

SEQ ID NO: 27 - DNA, 897 bp, synthetic construct
  source 1..897: mol_type = other DNA
gtggccctgcaggtcgccagtgcgccccacggcagcagcgaggacagcacctcggcgagcctgccggcgaactatccctatcacacgcggatgcgccgcaacgagtacgagaaggccaagcacgacctgcagatcgaactgctcaaggtgcagagctgggtgaaggagaccggccagcgcgtggtggtcctgttcgaaggccgcgacgccgccggcaagggcggcaccatcaagcgcttcatggaacacctgaacccgcgcggcgcgcggatcgtagccttggagaaaccgtcctcccaggagcagggccagtggtatttccagcgctacatccaacatctgcccaccgccggcgagatggtcttcttcgaccgctcctggtacaaccgcgccggcgtcgagcgggtcatgggcttctgttcgccgctgcaatacctggagttcatgcgccaggcgccggagctggagcgcatgctcaccaacagcggcatcctgctgttcaagtactggttctcggtgagccgcgaggaacaactgcggcgcttcatctcgcgccgcgacgatccgctcaagcactggaagctgtcgcccatcgacatcaagtctctggacaagtgggacgactacaccgccgccaagcaggcgatgttcttccataccgacaccgccgacgcgccgtggacggtcatcaagtccgacgacaagaagcgcgcgcgactcaactgcatccgccacttcctgcactcgctggactacccggacaaggaccggcgcatcgcccatgagcccgacccgttgctggtggggccggcctcgcgggtgatcgaggaggacgagaaggtctacgccgaggcggccgccgcgccgggccacgcgaacctggatatcccggcctaa

SEQ ID NO: 28 - DNA, 921 bp, synthetic construct
  source 1..921: mol_type = other DNA
atggtgggtaaacttcccatcatggctgaaaccaacgaaaatgatcttccagttatcgaccttgcccaaatcgaaggctatgttgtagatgactcggatgaagatgatccagtacttctgcgtccagatggaacccccattgaaacctggcgcgaagacttcccttatgaagagcgcgtcacccgcgaagactatgagaaggtcaagcgctccctccagatcgagctgctgaagtggcagaactggaccaaggaaactggccagcgccacatcattttgttcgaagggcgtggagccgctggtaagggtggcaccattaagcgcttcaacgaacacctgaaccctcgtggtgcccgtactgttgcgttggagaagccatcaccacgcgaatccacctcatggtacttccagcgctatattcagcacttcccagctgctggcgagatcgttttctttgaccgctcttggtacaaccgttccggcgtggagcgcgtcatgggtttctgcaccgaatcacagcatgcagagttcctgcgtgaggttccaatgctggaaaacatgatcctgggctctggtatcagcttgaccaagttctggttctcggtgacccgtaaagagcagcgcacccgttttgctatccgccaggttgatcctgtgcgtcagtggaagctttccccaatggacttggcttcacttgatcgctgggatgattacacccgcgctaaggaagagcagttccgttacaccgacactgatgagtccccgtggatcaccatcaagtcgaatgacaagaagcgtgcgcgtatcaacgcgatgcgttatgtattgtccaagtttgattacaccgacaaggattacgagctcgttggtgagcctgaccctaaggttgtgcttcgtgggcgcgaccagatcggtgactag

SEQ ID NO: 29 - DNA, 741 bp, synthetic construct
  source 1..741: mol_type = other DNA
atgttagatcaagtatgccagcttgcacggaatgcaggcgatgccattatgcaggtctacgacgggacgaaaccgatggacgtcgtcagcaaagcggacaattctccggtaacggcagcggatattgccgctcacaccgttatcatggacggtttacgtacgctgacaccggatgttccggtcctttctgaagaagatcctcccggttgggaagtccgtcagcactggcagcgttactggctggtagacccgctggatggtactaaagagtttattaaacgtaatggcgaattcaccgttaacattgcgctcattgaccatggcaaaccgattttaggcgtggtgtatgcgccggtaatgaacgtaatgtacagcgcggcagaaggcaaagcgtggaaagaagagtgcggtgtgcgcaagcagattcaggtccgcgatgcgcgcccgccgctggtggtgatcagccgttcccatgcggatgcggagctgaaagagtatctgcaacagcttggcgaacatcagaccacgtccatcggctcttcgctgaaattctgcctggtggcggaaggacaggcgcagctgtacccgcgcttcggaccaacgaatatttgggacaccgccgctggacatgctgtagctgcagctgccggagcgcacgttcacgactggcagggtaaaccgctggattacactccgcgtgagtcgttcctgaatccggggttcagagtgtctatttactaa

SEQ ID NO: 30 - DNA, 822 bp, synthetic construct
  source 1..822: mol_type = other DNA
atgaggccggtgccttggggcgaattggtggcgctggtgcggcgcgccggcgaggcgatcctgccgcactggcgcgccgacgtggtggtgcgctcgaaggccgacgaatcgccggtgactgccgccgacctggccgcgcaccatatattggaggcgggattgcgggcgctggcgccggacattccggtgctttccgaagaggattgcgagataccgctgagcgagcgcggccactggcggcgctggtggctggtggacccgctggacggcaccaaggagttcatctccggtagcgaggagttcaccgtcaacgtggccctggtcgaggatggccgggtgctgttcggcctggtcggcgtgccggtgagcggccgctgctactacggtggcgccggtctcggtgcctggcgcgaggaggccgatggccgcgcgcaaccgatcagtgtgcgcctggagcccgaggaggccttcaccgtggtggccagcaagcgccatggcagcccggcccaggagcgcctgctggatggcttgagcgagcgcttcggcgacctgcggcgagccagcatcggcagttcgctgaagttctgcctgctggccgagggcgctgccgactgctatccgcgcctgacgccaacctcgcaatgggacacggccgccgcccagggtgtgctggaaggcgccggcggcgaggtgctcgacctgcatggtgcgccattcacctacgagccgcgcgaggattacctcaacggctccttcctggccctgccgcgcgccgccgagtggcgcagcgagctgatccaactggcgcgcgcgctgcactga